(19) United States
(12) Patent Application Publication
(10) Pub. No.: US 2025/0090776 A1
(43) Pub. Date: Mar. 20, 2025

(54) COMPACT INHALER MECHANISM

(71) Applicant: RECIPROCAL LABS CORPORATION, San Diego, CA (US)

(72) Inventor: Robert Louis Baddeley, San Diego, CA (US)

(21) Appl. No.: 18/727,630

(22) PCT Filed: Jan. 10, 2023
(86) PCT No.: PCT/US2023/010459

§ 371 Date: Jul. 9, 2024

Related U.S. Application Data
(60) Provisional application No. 63/298,142, filed on Jan. 10, 2022.

(51) Int. Cl. A61M 15/00

(52) U.S. Cl. CPC A61M 15/009; A61M 2205/3358; A61M 2205/43

(57) ABSTRACT

A compact inhaler for dispensing medicament is disclosed. The compact inhaler includes a cylindrical main body housing having a mouthpiece, an open end, and an activation button aperture. A canister is stored in the mouthpiece. An activation button is moveable in the button aperture. Pressing the activation button pushes the canister out of the mouthpiece. The stored canister may be inserted in the open end of the main body to provide medicament from the mouthpiece when the canister is actuated.

### CROSS-REFERENCE TO RELATED APPLICATIONS

This application claims the benefit of, and priority to, U.S. Provisional Patent Application No. 63/298,142 filed Jan. 10, 2022, which is hereby incorporated by reference herein in its entirety.

### TECHNICAL FIELD

The present disclosure relates generally to medicament inhalers, and more specifically for a compact inhaler mechanism for a single use.

### BACKGROUND

Currently, many patients with ailments are provided an inhaler to provide dosages of medication. For example, an asthma patient may be provided a stimulant to assist mucus and reducing inflammation in clearing up breathing passages. Thus, when the patient experiences asthma exacerbations or as a daily maintenance medication to control symptoms, the patient can put the inhaler in front of their mouth and activate the inhaler spray, delivering a puff or puffs of the drug into the lungs in order to relieve the symptom.

A known pressurized metered dose inhaler (pMDI) inhaler, such as the AstraZeneca Symbicort Rapihaler®, for inhalation of drugs has an actuator housing at least partially defining a flow passageway extending through the inhaler from an air inlet to an outlet. A pressurized metered dose canister is held by the actuator. The canister includes a valve stem and a metering valve arranged to seat in a valve stem block formed on the housing and a main canister body of the canister may be moved relative to the housing and valve stem so as to operate the metering valve and fire a metered puff of propellant and active drug through the valve stem block and into the flow passageway. By depressing the canister, when a user inhales through a mouthpiece of the housing, air may be drawn into the housing between the canister and an inner wall of the housing, and may flow along past the canister towards the outlet. The Symbicort Rapihaler® delivers a combination of budesonide and formoterol (ICS/LABA combination) to treat asthma and/or chronic obstructive pulmonary disease (COPD). Other types of inhalers may deliver other kinds of medicaments for such ailments and other ailments. Such inhalers include the Teva Redihaler®.

In most known inhaler, the canister is placed in a holder but the mouthpiece remains accessible to catch debris between uses. One significant problem is users who use the inhalers infrequently. When inhalers are not cleaned regularly or properly, the mouth pieces may be clogged or accumulate debris. The canisters also have multiple doses and are not efficient in cases of emergency use, which require a single dose.

There is a need for a compact inhaler that allows the easy administration of a medicament. There is a need for a compact inhaler that protects the mouthpiece until the inhaler is deployed by a user. There is also another need for a compact inhaler that may be carried conveniently by a user.

### SUMMARY

One disclosed example is an inhaler including a main body housing having a mouthpiece, an open end, and an activation button aperture. The mouthpiece is operable to store a medicament canister. An activation button is moveable in the button aperture. Pressing the activation button pushes a stored medicament canister out of the mouthpiece. A medicament canister may be inserted in the open end of the main body to provide medicament from the mouthpiece when actuated.

A further implementation of the example inhaler is an embodiment that includes a cover that is fit over the open end. Pushing the activation button releases the cover. Another implementation is where the cover includes a tab. The activation button includes a prong extending through the main body to contact the tab of the cover, and a canister stored in the mouthpiece. Another implementation is where the canister includes a rescue medicament for a respiratory ailment. Another implementation is where the inhaler includes a key ring hole formed through the main body housing. Another implementation is where the canister contains a single dose of the medicament. Another implementation is where the main body includes a closed end opposite the open end. The closed end includes a stem for mounting a spray stem of the canister. Another implementation is where the inhaler includes an adherence monitor that is configured to determine actuation of the canister via an actuation detection sensor. Another implementation is where the actuation detection sensor is a pressure sensor or a mechanical switch. Another implementation is where the adherence monitor is operable to apply a time stamp to collected data indicating actuation of the inhaler. Another implementation is where the actuation detection sensor detects inhalation of the medicament. Another implementation is where the adherence monitor includes a sound generation device and a transceiver. The transceiver is operable to receive a signal from an external device to activate the sound generation device.

Another example is a compact inhaler including a cylindrical main body housing having a mouthpiece, an open end, and an activation button aperture. A canister is stored in the mouthpiece. An activation button is moveable in the button aperture. Pressing the activation button pushes the canister out of the mouthpiece. The stored canister may be inserted in the open end of the main body to provide medicament from the mouthpiece when the canister is actuated.

A further implementation of the example inhaler is an embodiment including a cover that is fit over the open end. Pushing the activation button releases the cover. Another implementation is where the canister includes a rescue medicament for a respiratory ailment. Another implementation is where the inhaler includes a key ring hole formed through the main body housing. Another implementation is where the main body includes a closed end opposite the open end. The closed end includes a stem for mounting a spray stem of the canister. Another implementation is where the inhaler includes an adherence monitor that is configured to determine actuation of the canister via an actuation detection sensor.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims.

### BRIEF DESCRIPTION OF THE DRAWINGS

The disclosure will be better understood from the following description of exemplary embodiments together with reference to the accompanying drawings, in which:

FIG. 1 is a perspective view of an example compact inhaler;

FIG. 2A is an exploded perspective view of the components of the example compact inhaler shown in FIG. 1;

FIG. 2B is a side exploded view of the example compact inhaler shown in FIG. 1;

FIG. 2C is a side cutaway exploded view of the example compact inhaler shown in FIG. 1;

FIG. 3A is a perspective view of the cap of the compact inhaler in FIG. 1;

FIG. 3B is a perspective view of the activation button of the compact inhaler in FIG. 1;

FIG. 3C is a perspective view of the main body of the compact inhaler in FIG. 1;

FIG. 3D is a perspective view of a one use canister for the compact inhaler in FIG. 1;

FIG. 4A shows a side cutaway view of the assembled components of the example compact inhaler in FIG. 1 in a storage mode;

FIG. 4B shows a top cutaway view of the assembled components of the example compact inhaler in FIG. 1 in the storage mode;

FIG. 5A is a side view showing the removal of the dosage canister from the example compact inhaler in FIG. 1 for use;

FIG. 5B is a side view showing the installation of the canister in the compact inhaler for administration of a medicament;

FIG. 5C is a front view showing the example compact inhaler when the canister is inserted and the inhaler is ready to administer a puff of the medicament;

FIG. 6 is a perspective view of the compact inhaler when attached to a key ring; and

FIG. 7 is a block diagram of the electronic components of an adherence/event detection module that may be embedded in the compact inhaler in FIG. 1.

The present disclosure is susceptible to various modifications and alternative forms. Some representative embodiments have been shown by way of example in the drawings and will be described in detail herein. It should be understood, however, that the invention is not intended to be limited to the particular forms disclosed. Rather, the disclosure is to cover all modifications, equivalents, and alternatives falling within the spirit and scope of the invention as defined by the appended claims.

### DETAILED DESCRIPTION OF THE ILLUSTRATED EMBODIMENTS

The present inventions can be embodied in many different forms. Representative embodiments are shown in the drawings, and will herein be described in detail. The present disclosure is an example or illustration of the principles of the present disclosure, and is not intended to limit the broad aspects of the disclosure to the embodiments illustrated. To that extent, elements and limitations that are disclosed, for example, in the Abstract, Summary, and Detailed Description sections, but not explicitly set forth in the claims, should not be incorporated into the claims, singly or collectively, by implication, inference, or otherwise. For purposes of the present detailed description, unless specifically disclaimed, the singular includes the plural and vice versa; and the word “including” means “including without limitation.” Moreover, words of approximation, such as “about,” “almost,” “substantially,” “approximately,” and the like, can be used herein to mean “at,” “near,” or “nearly at,” or “within 3-5% of,” or “within acceptable manufacturing tolerances,” or any logical combination thereof, for example.

The present disclosure relates to a compact inhaler that may be easily accessible to provide a puff of medicament such as a rescue medicament for a respiratory ailment. The example compact inhaler may be carried by a user for extended periods of time before use. The compact inhaler has a storage configuration that stores the canister of medicament in a mouthpiece of a main housing. A canister cylinder is plugged with a cap in the storage configuration. When a user desires to use the compact inhaler, the user pushes an activation button that releases the cap and the canisters. The cap may then be removed to access an opening in the main housing. The user may then remove the canister from the mouthpiece and put the canisters in the main housing. Once inserted, the user can push down on the canisters to administer the medicament through the now accessible mouthpiece.

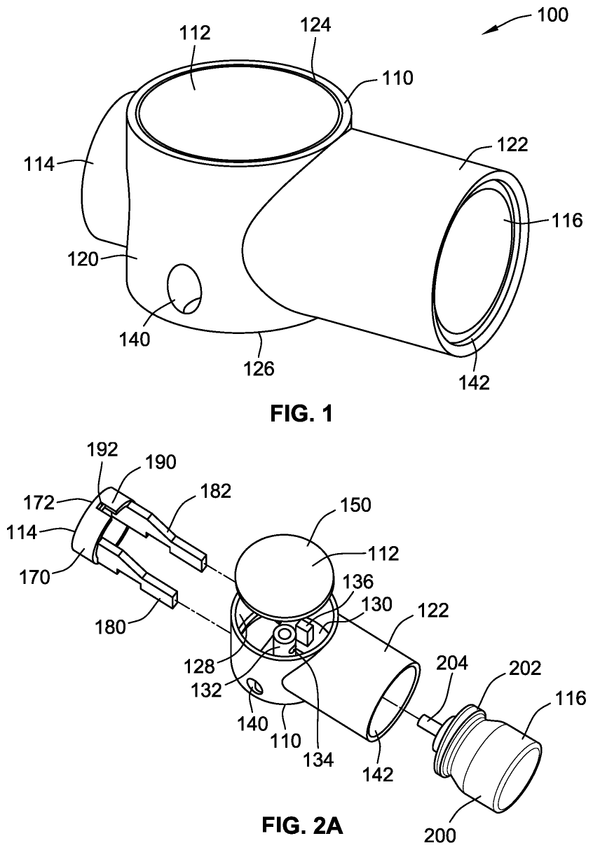

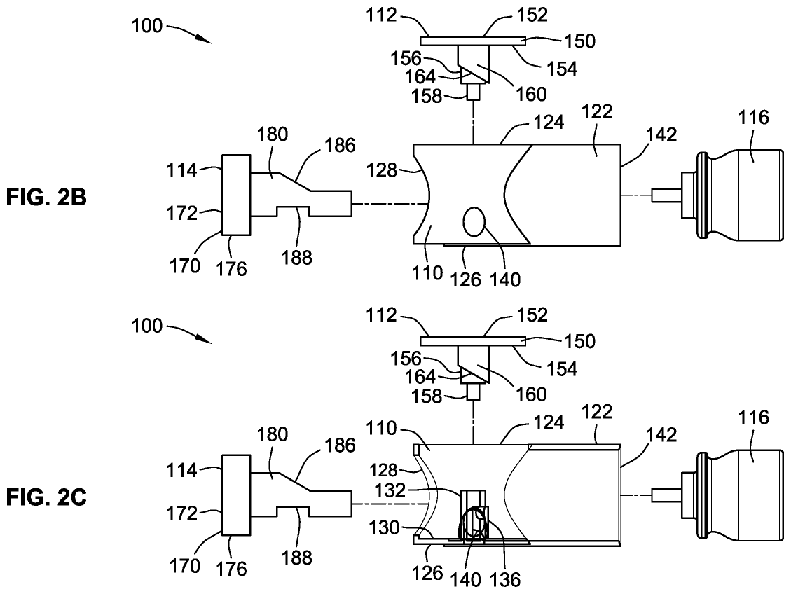

FIG. 1 is a perspective view of an example compact inhaler 100 that may be made easily accessible to a user for efficient administration of medicament. FIG. 2A is an exploded view of the components of the compact inhaler 100. FIG. 2B is a side exploded view of the components of the compact inhaler 100. FIG. 2C is an opposite side exploded view of the components of the compact inhaler 100. As shown in FIGS. 1-2, the example inhaler 100 includes a main body 110, a cover 112, an activation button 114, and a dosage canister 116. FIGS. 1-2 show the inhaler 100 in a storage mode where the dosage canister 116 is stored in the main body 110. The cover 112 is attached to the main body 110. The compact inhaler 100 may be carried with all the components stored in the main body 110.

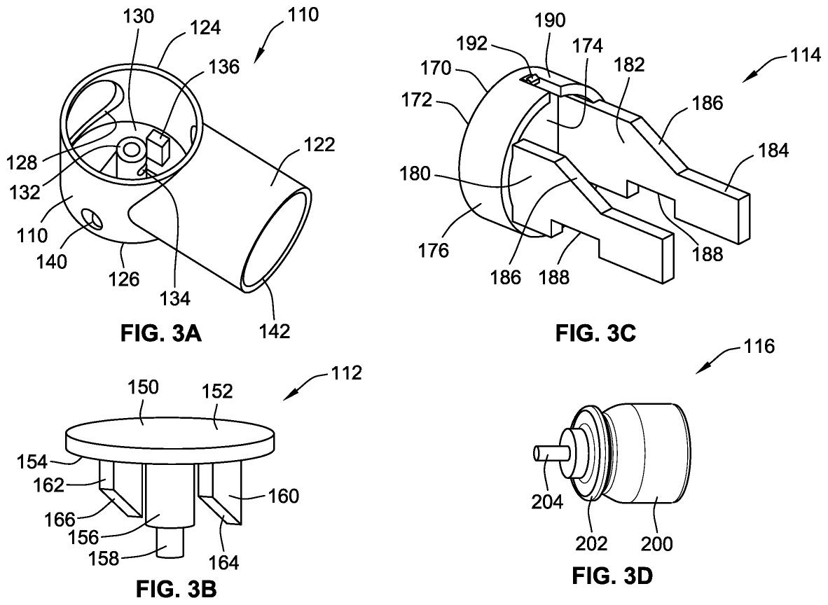

FIG. 3A is a perspective view of the main body 110 of the compact inhaler 100 in FIG. 1. The main body 110 has a cylindrical housing 120 with a connected mouthpiece 122. In this example, the housing 120 and the mouthpiece 122 are molded from plastic or similar materials such as ABS, polycarbonate, PETE, acrylic, or polystyrene. The mouthpiece 122 is cylindrically shaped and is of sufficient diameter to store the dosage canister 116. The mouthpiece 122 extends perpendicularly from the cylindrical housing 120 and allows access to the interior of the cylindrical housing 120. The cylindrical housing 120 has an open end 124 and an opposite closed end 126. The cylindrical housing 120 includes a circular outer surface between the open end 124 and the closed end 126. An opposite side of the cylindrical housing 120 from the mouthpiece 122 has a circular activation aperture 128 that holds the button 114.

The cylindrical housing 120 includes a bottom interior surface 130 that is enclosed by the cylindrical wall that forms the cylindrical housing 120. A stem 132 extends from the interior surface 130 of the closed end 126. The stem 132 is cylindrical and includes an open end and a circular opening 134 on the body of the stem 132. A pair of rectangular stubs 136 also extend from the interior surface 130 of the closed end 126. The stubs 136 are positioned on either side of the stem 132 and provide a registration feature for the activation button 114 as will be explained below. A keyhole 140 is cut through the side of the housing 120 and the closed end 126. The keyhole 140 allows the inhaler 100 to be attached to other objects such as a key ring. The mouthpiece 122 has an open proximal end 142 that allows either the canister 116 to be inserted, or when the canister 116 is removed, allows medicament to be inhaled by a user from the cylindrical housing 120. The mouthpiece 122 is of a sufficient length to enclose the entire canister 116 when the canister 116 is inserted through the open proximal end 142.

FIG. 3B is a perspective view of the cover 112 that functions to enclose the cylindrical housing 120 when the compact inhaler 100 is not in use. The cover 112 includes a cylindrical plate 150 that fits in the circular open end 124 of the cylindrical housing 120. The cylindrical plate 150 has a flat exterior surface 152 and an opposite interior surface 154. A central cylinder 156 extends downward from the interior surface 154 and supports a pin 158. The pin 158 may be inserted into the open end of the stem 132. In this example, the pin 158 is friction fit to the interior of the stem 132 to hold the cover 112 in place relative to the cylindrical housing 120.

A pair of tabs 160 and 162 extend down from the interior surface 154 of the cylindrical plate 150. The tabs 160 and 162 are spaced equidistance from the central cylinder 156 and are parallel to each other. Each of the tabs 160 and 162 have a general rectangular shape with a respective sloped surface 164 and 166. Certain registration features may be cut into the plate 150 and the corresponding open end 124 to insure that the cover 112 is aligned in a single orientation allowing the tabs 160 and 162 to contact prongs of the activation button 114 (as will be described below) when the cover 112 is inserted in the open end 124.

FIG. 3C is a perspective view of the activation button 114 that is held in the aperture 128 of the cylindrical housing 120 of the main body 110. The activation button 114 includes a cylindrical body 170 having a flat surface 172 that may be pushed into the cylindrical housing 120 by a user. Pushing the activation button 114 releases the cover 112 to provide access to the open end 124 of the cylindrical housing 120. Pushing the activation button 114 also releases and the canister 116 from the mouthpiece 122 of the main body 110. As will be explained, a user may the place the canister 116 in the open end 124. The cylindrical body 170 has a circular outer wall 176 having a diameter that allows the activation button 114 to be inserted through the aperture 128 of the housing body 110 of the main body 110. The cylindrical body 170 has an opposite inner surface 174 that includes two prongs 180 and 182. Both of the prongs 180 and 182 extend from the inner surface 174. The prongs 180 and 182 each include a distal end 184, a sloped contact surface 186, and a bottom slot 188. The prongs 180 and 182 are of sufficient length to extend into the interior volume of the cylindrical housing 120. As will be explained, the physical features of the prongs 180 and 182 allow the button 114 to remain attached to the main body 110 and also engage the top cover 112 and the canister 116 when the inhaler 100 is prepared for administering a puff of the medicament.

A slot 190 is formed on the outer wall 176 of the cylindrical body 170 of the activation button 114. Another slot is formed on the outer wall 176 opposite of the slot 190. A clip member 192 is formed in the slot 190 and another clip member is formed in the opposite slot. The clip members 192 engage the aperture 128 and holds the activation button 114 in place when the activation button 114 is pushed into the aperture 128. This prevents the button 114 from moving backwards. Of course, if the inhaler 100 is designed for multiple uses, different mechanisms may be used to allow the button 114 to be retracted once a replacement canister is inserted.

FIG. 3D is a perspective view of the canister 116 that is stored in the mouthpiece 122 until the medicament is needed by the user. In this example, the canister 116 holds a single use of medicament, such as a rescue medication. For example, rescue medications such as Beclomethasone, fluticasone, ciclesonide, mometasone, budesonide, flunisolide, albuterol sulfate may be provided for asthma. Although an amount of medicament sufficient for a single use is contained in the canister 116 in this example, an amount of medicament allowing for multiple uses may be carried by the canister 116, if the inhaler 100 is designed to be used for more than one use. The canister 116 has a generally cylindrically shaped container 200 that holds the medicament. The interior volume of the container 200 is sealed off to contain the medicament. The container 200 has a cylindrical wall that tapers in to an annular collar 202. The annular collar 202 has a closed end that has a spray stem 204 extending into the container 200. One end of the spray stem 204 is attached to an internal spring in the container 200. Pushing the stem 204 into the container 200 pressurizes the medicament that is propelled to be emitted through the stem 204 when the stem 204 is released.

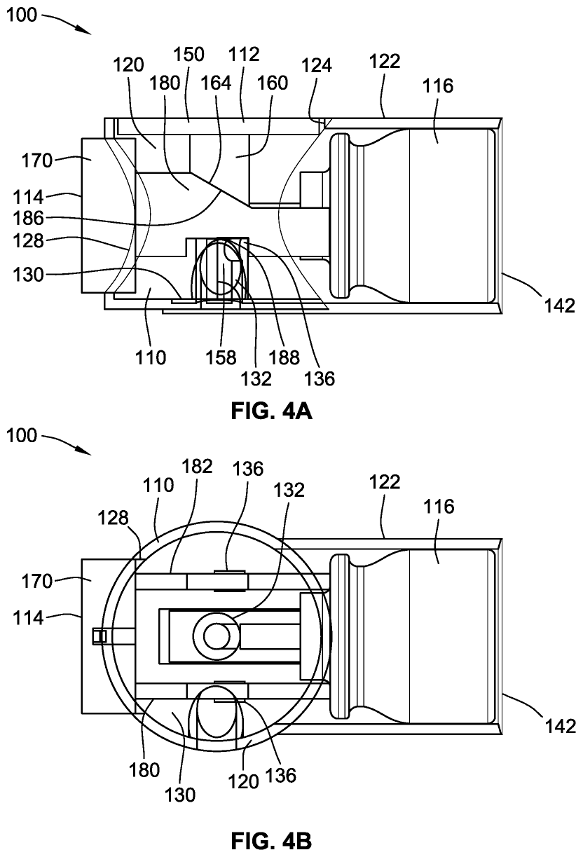

FIG. 4A shows a side cutaway view of the assembled components of the example compact inhaler 100 in the storage configuration. FIG. 4B shows a top cutaway view of the assembled components of the example compact inhaler 100 in the storage configuration. As shown in FIGS. 4A-4B, when in a storage configuration, the canister 116 is inserted through the open end 142 of the mouthpiece 122 so the mouthpiece 122 is substantially contained within the mouthpiece 122. The canister 116 thus is stored in the mouthpiece 122 and thus protects the mouthpiece 122 from debris that may enter through the open end 142. The mouthpiece 122 also protects the canister 116 from any external debris. The canister 116 is held in place in the mouthpiece 122 via friction fit or by contact with a small ridge in the mouthpiece 122 providing a friction contact point. Alternatively, the canister 116 may be sealed in the mouthpiece 122 by foil. The canister 116 is readily accessible by the user when it is stored in the mouthpiece 122.

The cylindrical plate 150 of the cover 112 is shaped to fit within the circular open end 124 of the housing 120. The pin 158 is inserted in the opening of the stem 132 that extends from the interior surface 130 of the closed end 126. The pin 158 may be friction fit within the stem 132 to hold the cover 112 in place in the open end 124. Thus, the cover 112 protects the interior of the housing 120 from debris entering from the open end 124 in the storage configuration.

The cylindrical body 170 of the activation button 114 extends outward from the aperture 128 of the main housing 120. When the activation button 114 is in an extended position, one edge of the bottom slots 188 of the prongs 180 and 182 rest against the edge of the respective stubs 136 extending from the bottom interior surface 130 of the closed end 126 of the housing 120. When the activation button 114 is in an extended position, the sloped contact surfaces 186 of the prongs 180 and 182 rest against the corresponding sloped surfaces 164 and 166 of the tabs 160 and 162 of the cover 112.

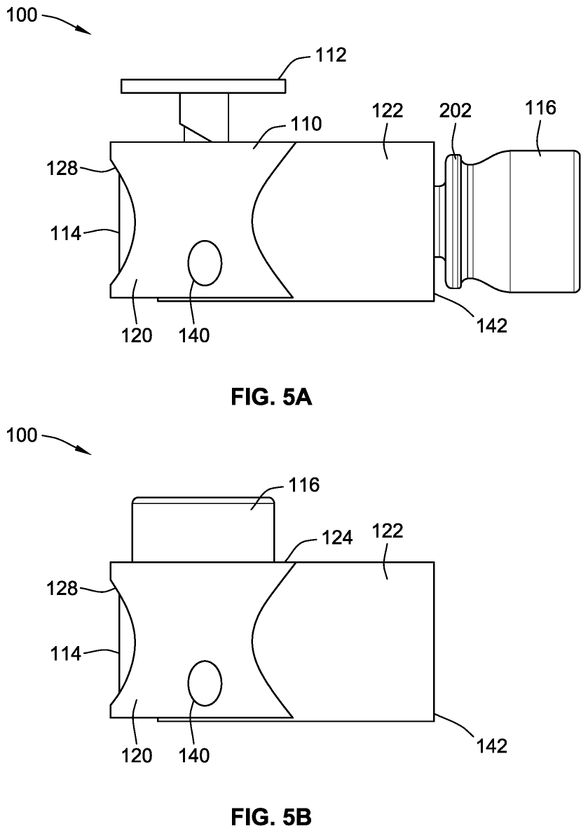

FIG. 5A is a side view showing the process of removing the dosage canister 116 from the main body 110 for use. When a user desires to use the inhaler 100, the user pushes the activation button 114 into the housing 120 through the aperture 128. When the activation button 114 is pushed, the cylindrical body 170 moves into the cylindrical housing 120 of the main body 110. Pushing the button 114 pushes the cover 112 upward and thereby releases the cover 112 from the open end 124. When the button 114 is pushed, the prongs 180 and 182 are moved and the sloped contact surfaces 186 push against the sloped surfaces 164 and 166 of the tabs 160 and 162 and thus force the cover 112 upward. The button 114 is limited in its movement into the cylindrical housing 120 by the opposite edges of the slots 188 contacting the stubs 136. Thus, the stubs 136 serve as a stop, preventing the button 114 from being pushed further into the cylindrical housing. Once the cover 112 is pushed up over the open end 124 by the prongs 180 and 182, the user may then remove the cover 112. The cover 112 may either be discarded or saved by the user if a replacement canister 116 is provided.

The distal ends of the prongs 180 and 182 push against the annular collar 202 of the canister 116. The canister 116 thus moves out from the open end 142 of the mouthpiece 122. The user may thus remove the canister 116 from the open end 142 of the mouthpiece 122 once the canister 116 is partially pushed out by the prongs 180 and 182.

The now free canister 116 may then be inserted in the open end 124 of the housing 120 of the main body 110 since the cover 112 has been removed. FIG. 5B is a side view showing the installation of the canister 116 in the main body 110 for administration of a medicament. FIG. 5C is a front view showing the example compact inhaler 100 when the canister 116 has been inserted in the open end 124 of the housing 120 and is therefore ready to administer a puff. When the canister 116 is lowered into the open end 124, the stem 204 of the canister 116 is inserted in the hole of the stem 134. Thus, the canister 116 is seated in the cylindrical housing 120 and is ready for activation.

To dispense a puff, the user holds the inhaler 100 and puts the mouthpiece 122 in their mouth. The user may then push the canister 116 downward in the housing 120. The downward motion of the canister 116. When the canister 116 is pushed down, it contacts an internal actuator stem, which compresses an internal spring to cause pressure to force the stored medicament out of the spray stem 204 through the opening 134 in the stem 132. The spray stem 204 in the canister 116 and the internal actuator stem thus combine to deliver a metered puff of medicament. The medicament is forced out through the open end 142 of the mouthpiece 122 into the user's throat out through the mouthpiece 122, and into the mouth of the user. Ideally, a user will push down the canister 116 at the same time the user inhales, thus maximizing the inhalation of the medicament dose. The canister 116 ideally contains sufficient medicament for multiple puffs to address an emergency event.

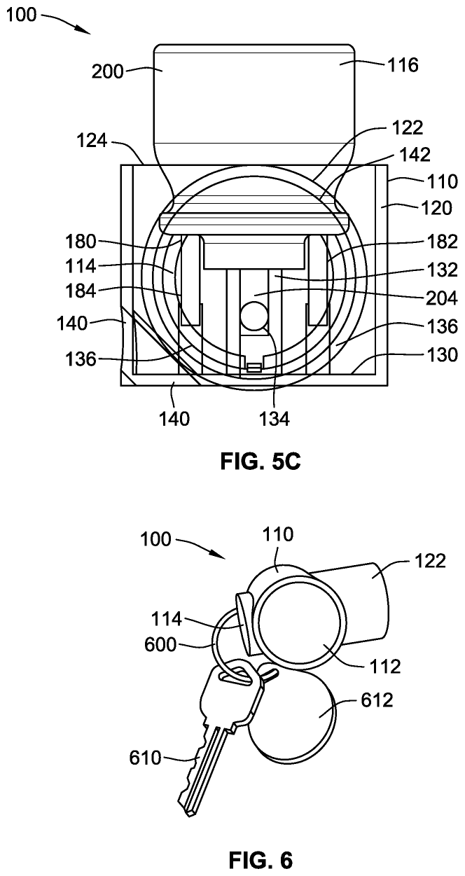

The inhaler 100 is designed to be carried by a user in the storage mode as shown in FIGS. 4A-4B until a dosage is required. The keyhole 140 formed through the housing 120 may be used to attach the inhaler 100 to another object such as a key ring. FIG. 6 is a perspective view of the example compact inhaler 100 when attached to a key ring 600. The key ring 600 has an end that forms a circle. One end may be inserted through the hole 140 to attach the inhaler 100 to the key ring 600 along with other objects such as keys 610 or a fob 612. The user may thus carry the compact inhaler 100 in a pocket or any other convenient carrying place. The user may therefore have ready access to the inhaler 100 should there be a need for a medicament dosage.

The compact inhaler 100 may be for a single use where the canister 116 is packaged with the compact inhaler. Alternatively, the main body 110, cover 112, activation button 114, may be used with a refill canister similar to the canister 116. Thus, after a use, the user may discard the canister 116 or place the empty canister 116 back into the mouthpiece 122, and replace the cover 112. The user may then insert a fresh canister 116 into the mouthpiece 122 when one is convenient and reuse the compact inhaler 100.

An adherence/event detection module may be embedded or attached to the compact inhaler 100. The adherence/event detection module allows detection of use of the compact inhaler 100 and may allow recording the use as well as transmitting data to an external device or system for purposes such as compliance, treatment or diagnosis in relation to respiratory ailments.

In this example, the adherence/event detection module detects when the user presses down on the canister 116 and captures a timestamp of the actuation event in on board non-volatile (NV) memory. Other data on inhalation may be collected and added to the timestamp of the event. The adherence/event detection module may then advertise a connection using a transmission protocol such as Bluetooth Low Energy (BLE) in order to establish a link to a client device such as a smart phone. Once a BLE link is formed, the adherence/event detection module will send any event records (inhalations) to the client device for further analysis of adherence in relation to using the compact inhaler 100.

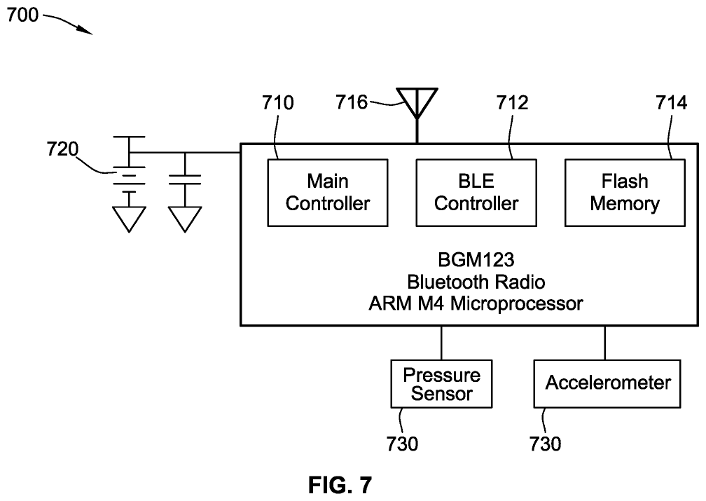

FIG. 7 is block diagram of an example adherence monitor module 700 that may be mounted in the housing 120. The monitor module 700 includes a controller 710, internal memory 712, and a transmitter 714. Data signals are transmitted via an antenna 716. The monitor module 700 may be powered by a battery 720. A series of sensors may be embedded in the module 700 or located in appropriate registration features in the housing 120. In this example, the sensors include a barometric pressure sensor 730 and an accelerometer 732. In this example, actuation of the canister 116 may be detected by a change in air pressure detected by the barometric pressure sensor 730.

Alternatively, a simple mechanical switch triggered by the motion of the canister could detect actuation and send a signal to the controller 710. Another switch could also detect pressing the activation button, removal of the cap, or removal of the canister from the mouthpiece, any of which would signal that the device had been prepared for use to the controller 710. A sound generation device such as a piezoelectric buzzer or magnetic speaker element may be added within the monitor module 700 to enable a feature of locating a lost inhaler. By locating a Bluetooth Low Energy system that may be part of the controller 710 with an external device a smartphone or other capable device, the speaker can be activated via a signal received by the BLE system to produce loud tones, allowing the user to find the compact inhaler 100.

In this example, the controller 710 executes different firmware for collecting data. The memory 712 stores firmware and data storage. In this example the controller 710 includes Bluetooth Low Energy (BLE) system in package (SIP). Of course any suitable component or set of components with appropriate functionality may be used for the communication module 316. For example, a BGM123 BLE SIP includes an ARM M4 microcontroller and an ARM M0 microcontroller (controller 714) that run the internal Bluetooth Smart™ compliant stack. In this example, the BGM123 BLE SIP acts as both the Bluetooth radio and the main microprocessor of the adherence monitor module 700. In this example, the memory 712 may be embedded flash memory in the BGM123 BLE SIP that stores the firmware that is executed on the controller 710. A portion of this flash memory is also used to store the event records that are detected by the various sensors 730 and 732.

The BGM123 BLE SIP also contains embedded SRAM memory used for temporary scratch space and data structures. Several peripherals (UART, SPI, A2D, RTC, PWM, DMA, and power management) are utilized to drive the system. The BGM123 BLE SIP is driven directly from the onboard battery 720 with no other power regulation other than its internal DC-DC switcher which it uses to improve the energy efficiency of the system. The module contains the integrated omni-directional chip antenna 716 that allows transmission of the collected data to an external client device.

Usage events contain a timestamp captured from the moment that the barometric pressure sensor 730 detects actuation of the canister 116. If the accelerometer 732 detects shaking prior to actuation, the shake intensity and shake duration are added to the usage event. If the barometric pressure sensor 730 detects an inhalation around the time of actuation, either before, during, or after, the peak value of the pressure measurement, the duration of inhalation, and the time between the start of inhalation to the peak of inhalation are added to the usage event. All usage events will also include a battery measurement and temperature measurement captured from the controller at the time of actuation.

The adherence monitor module 700 collects data on inhaler actuation and provides time stamp and other related data. The collected data includes an inhaler actuation event with a unique identifier, a time stamp for the event, and sensor battery level.

In addition, based on the knowledge of the sensor type, and its association by the user with the medicament via a user interface, the medicament may be known and added to the data record by either the external device or an external server that receives data from the adherence monitor. Additional data such as the number of puffs, may also be appended to the data record by the external device or the external server. Finally, individual sensors collect additional data specific to the sensor. The example adherence monitors collect the shake duration and intensity of a shake of the medicament prior to use. The barometric pressure sensor 730 may allow collecting the peak value of the inhalation and the duration of the inhalation. The example adherence monitor module 700 may collect the time of peak as difference between inhalation start and peak value, as well as total volume of inhalation. The adherence monitor module 700 captures the time between actuation (when the medicament is released) and inhalation start, as well as the number of seconds the medicament actuator is pressed. With this data, a health care provider may determine whether the patient is using their medicament as instructed with proper inhalation technique. By evaluating trends in this data, health care providers can provide instruction on more effective use of the medication, or may determine that changes to their treatment are necessary.

As used in this application, the terms “component,” “module,” “system,” or the like, generally refer to a computer-related entity, either hardware (e.g., a circuit), a combination of hardware and software, software, or an entity related to an operational machine with one or more specific functionalities. For example, a component may be, but is not limited to being, a process running on a processor (e.g., digital signal processor), a processor, an object, an executable, a thread of execution, a program, and/or a computer. By way of illustration, both an application running on a controller, as well as the controller, can be a component. One or more components may reside within a process and/or thread of execution, and a component may be localized on one computer and/or distributed between two or more computers. Further, a “device” can come in the form of specially designed hardware; generalized hardware made specialized by the execution of software thereon that enables the hardware to perform specific function; software stored on a computer-readable medium; or a combination thereof.

The terminology used herein is for the purpose of describing particular embodiments only, and is not intended to be limiting of the invention. As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof, are used in either the detailed description and/or the claims, such terms are intended to be inclusive in a manner similar to the term “comprising.”

Unless otherwise defined, all terms (including technical and scientific terms) used herein have the same meaning as commonly understood by one of ordinary skill in the art. Furthermore, terms, such as those defined in commonly used dictionaries, should be interpreted as having a meaning that is consistent with their meaning in the context of the relevant art, and will not be interpreted in an idealized or overly formal sense unless expressly so defined herein.

One or more elements or aspects or steps, or any portion(s) thereof, from one or more of any of claims 1 to 18 below can be combined with one or more elements or aspects or steps, or any portion(s) thereof, from one or more of any of the other claims 1 to 18 or combinations thereof, to form one or more additional implementations and/or claims of the present disclosure.

While various embodiments of the present invention have been described above, it should be understood that they have been presented by way of example only, and not limitation. Although the invention has been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application. Thus, the breadth and scope of the present invention should not be limited by any of the above described embodiments. Rather, the scope of the invention should be defined in accordance with the following claims and their equivalents.

1. An inhaler comprising:
a main body housing having a mouthpiece, an open end, and an activation button aperture, wherein the mouthpiece is operable to store a medicament canister; and
an activation button moveable in the button aperture, wherein pressing the activation button pushes a stored medicament canister out of the mouthpiece, and wherein a medicament canister may be inserted in the open end of the main body to provide medicament from the mouthpiece when actuated.

2. The inhaler of claim 1, further comprising a cover that is fit over the open end, wherein pushing the activation button releases the cover.

3. The inhaler of claim 2, wherein the cover includes a tab, and the activation button includes a prong extending through the main body to contact the tab of the cover, and a canister stored in the mouthpiece.

4. The inhaler of claim 1, wherein the canister includes a rescue medicament for a respiratory ailment.

5. The inhaler of claim 1, further comprising a key ring hole formed through the main body housing.

6. The inhaler of claim 1, wherein the canister contains a single use of the medicament for a rescue event.

7. The inhaler of claim 1, wherein the main body includes a closed end opposite the open end, wherein the closed end includes a stem for mounting a spray stem of the canister.

8. The inhaler of claim 1, further comprising an adherence monitor that is configured to determine actuation of the canister via an actuation detection sensor.

9. The inhaler of claim 8, wherein the actuation detection sensor is one of a barometric pressure sensor or a mechanical switch.

10. The inhaler of claim 8, wherein the adherence monitor is operable to apply a time stamp to collected data indicating actuation of the inhaler.

11. The inhaler of claim 8, wherein the actuation detection sensor detects inhalation of the medicament.

12. The inhaler of claim 8, wherein the adherence monitor includes a sound generation device and a transceiver, wherein the transceiver is operable to receive a signal from an external device to activate the sound generation device.

13. A single use compact inhaler comprising:
a cylindrical main body housing having a mouthpiece, an open end, and an activation button aperture;
a canister stored in the mouthpiece; and
an activation button moveable in the button aperture, wherein pressing the activation button pushes the canister out of the mouthpiece, and wherein the canister may be inserted in the open end of the main body to provide medicament from the mouthpiece when the canister is actuated.

14. The inhaler of claim 13, further comprising a cover that is fit over the open end, wherein pushing the activation button releases the cover.

15. The inhaler of claim 13, wherein the canister includes a rescue medicament for a respiratory ailment.

16. The inhaler of claim 13, further comprising a key ring hole formed through the main body housing.

17. The inhaler of claim 13, wherein the main body includes a closed end opposite the open end, wherein the closed end includes a stem for mounting a spray stem of the canister.

18. The inhaler of claim 13, further comprising an adherence monitor that is configured to determine actuation of the canister via an actuation detection sensor.
